(19) United States
(12) Patent Application Publication
(10) Pub. No.: US 2002/0049414 A1
(43) Pub. Date: Apr. 25, 2002

(54) Fluid delivery and extraction device and method

(76) Inventors: Anthony A. Nobles, Fountain Valley, CA (US); Alan Eskuri, Hanover, MN (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/970,245

(22) Filed: Oct. 3, 2001

Related U.S. Application Data
(60) Provisional application No. 60/237,662, filed on Oct. 3, 2000.

(51) Int. Cl. A61M 5/00

(52) U.S. Cl. 604/187; 604/173

(57) ABSTRACT

A fluid delivery and extraction device enables the remote transfer of fluid to or from biological tissue. The device comprises an elongated member, a syringe movable relative to the elongated member and a pair of hypodermic needles movable relative to the elongated member. The elongated member has a distal portion which is adapted to be inserted into a biological structure. A steer wire or guide wire can be used to navigate the distal portion within cavernous biological structures, particularly body lumens. The distal portion has at least one retractable hypodermic needle that is configured to pierce the interior surface of a tubular biological structure and transfer fluid to or from the walls of the tubular biological structure. A physician can use a handle or other control mechanism provided at a proximal portion of the device to remotely move the hypodermic needles. A plunger can be used to transfer fluid through the hypodermic needles to or from the syringe. In operation, the hypodermic needles are deployed simultaneously or individually from the distal portion of the device. As the hypodermic needles move, they pierce the interior surface of the tubular biological structure. The hypodermic needles can also be configured to pass beyond the exterior surface of the tubular biological structure. The plunger is then used to transfer fluid either from the syringe to the patient or from the patient to the syringe. The hypodermic needles are then moved back to their retracted positions within the distal portion, and the device is withdrawn from the patient's body.

### CROSS-REFERENCE TO RELATED APPLICATION

This application claims the benefit of U.S. Provisional Application Serial No. 60/237,662, filed on Oct. 3, 2000, the entirety of which is hereby incorporated by reference.

### BACKGROUND OF THE INVENTION

1. Field of the Invention

The present invention relates generally to medical devices. Specifically, the present invention relates to a fluid delivery and extraction device and method for transferring fluid to or from biological tissue that may not be directly accessible.

2. Description of the Related Art

Invasive medical procedures typically cause trauma and tissue damage to the patient. Less invasive processes are ordinarily preferred, particularly when a biological structure to be medically treated is a body lumen, such as a blood vessel or a fallopian tube.

Medical catheters such as balloon catheters have been proven efficacious in treating a wide variety of blood vessel disorders. Moreover, these types of catheters have permitted clinicians to treat disorders with minimally invasive procedures that, in the past, would have required complex and perhaps life-threatening surgeries. For example, balloon angioplasty is now a common procedure to alleviate stenotic lesions (i.e., clogged arteries) in blood vessels, thereby reducing the need for heart bypass operations.

Unfortunately, roughly 30% of angioplasty patients experience restenosis (i.e., re-narrowing of a treated artery) following an angioplasty procedure. Restenosis occurs due to cell overgrowth following angioplasty procedures. This can lead to further angioplasty procedures or heart bypass surgery on an emergency basis. If not diagnosed and treated quickly enough, restenosis can lead to stroke or death.

A wide variety of approaches to preventing restenosis have been proposed. One noteworthy approach is the administration of cell growth inhibiting compounds directly within the artery being treated with angioplasty. Another approach is the administration of angiogenesis compounds within the treated artery. These approaches typically employ perforated angioplasty balloons to deliver compounds into the artery at the site of the angioplasty treatment.

Conventional fluid delivery devices, however, fail to provide a means by which fluid compounds can be remotely injected directly into body tissue, such as the walls of blood vessels or fallopian tubes.

### SUMMARY OF THE INVENTION

The preferred embodiments of the present invention describe a fluid delivery and extraction device that enables the remote transfer of fluid to or from biological tissue. In general, the fluid delivery and extraction device may advantageously reach biological structures that are typically unreachable or difficult to reach by use of other fluid delivery and extraction devices and methods. A steer wire or guide wire may be used to navigate a distal portion of the fluid delivery and extraction device within cavernous biological structures, particularly body lumens. One embodiment of the fluid delivery and extraction device is ideally suited for delivering fluid compounds to a tubular biological structure, such as a blood vessel or a fallopian tube. Another embodiment of the fluid delivery and extraction device is ideally suited for extracting fluid from biological structures that are not directly accessible, such as a tumor or colloid cyst.

One aspect of the invention relates to delivering fluid compounds to a biological structure having an interior surface. One preferred device comprises an elongated member that is adapted for insertion into and navigation within the biological structure. The device further comprises a luer connector movable relative to the elongated member, a syringe that seals to the luer connector, and a pair of hypodermic needles. Each hypodermic needle has a proximal end and a distal end. The proximal end is molded into the luer connector to permit fluid transfer between the hypodermic needle and the syringe. The hypodermic needles are mounted so as to move relative to the elongated member. The distal ends of the hypodermic needles are movable from a retracted position adjacent to the elongated member to an extended position spaced radially away from the elongated member. A handle mechanism provided at the proximal portion of the elongated member enables a user to remotely control the movement of the hypodermic needles. The distal ends of the needles are adapted to penetrate the interior surface of and transfer fluid to or from the biological structure. A plunger inserted into the proximal end of the syringe enables a user to transfer fluid through the hypodermic needles to or from the syringe.

In one embodiment of the invention, an anchor hook (i.e., a “J-hook”) is included on the distal portion of the fluid delivery and extraction device. The anchor hook serves as a means by which the distal portion can be anchored or approximated to desired tissue locations within biological structures, particularly body lumens. The anchor hook remains in a retracted position while the distal portion of the fluid and delivery and extraction device is advanced within the tubular biological structure. Once the distal portion reaches the desired location, the anchor hook may be remotely deployed and operated by use of control mechanisms on the proximal portion of the fluid delivery and extraction device. In one embodiment, the anchor hook may be substituted for one of the two hypodermic needles on the distal portion. In another embodiment, the anchor hook may be used in addition to the two hypodermic needles.

In another embodiment, an angioplasty balloon is included on the distal portion of the fluid delivery and extraction device. The angioplasty balloon is housed inside the distal portion of the fluid and delivery and extraction device while the distal portion is advanced within a tubular biological structure. Once the distal portion reaches the desired location, the angioplasty balloon may be remotely deployed and inflated by use of control mechanisms on the proximal portion of the fluid delivery and extraction device. In one embodiment, the angioplasty balloon may be used to anchor or approximate the distal portion to desired tissue locations within biological structures, particularly body lumens. In another embodiment, the angioplasty balloon may be used to dilate ischemic tissue or a stenosis as is commonly performed in angioplasty procedures.

In still another embodiment, a pair of biopsy jaws may be included on the distal portion of the fluid delivery and extraction device. The pair of biopsy jaws is housed in a retracted position within the distal portion while the distal portion is advanced within a biological structure. Once the distal portion is located at the desired position with the biological structure, the pair of biopsy jaws may be remotely deployed by control mechanisms on the proximal portion of the fluid delivery and extraction device. In one embodiment, the biopsy jaws may advantageously be used to perform biopsy procedures on or within biological structures that are not directly accessible. In another embodiment, the biopsy jaws may advantageously be used to anchor or approximate the distal portion to desired tissue locations within biological structures.

In yet another embodiment, the distal portion of the fluid delivery and extraction device may have four hypodermic needles, deployable in a manner substantially similar to the two-needle embodiment described above. In another embodiment, the distal portion may have six hypodermic needles, which can be deployed in a manner substantially similar to the two-needle embodiment described above. In still another embodiment, the distal portion may have eight hypodermic needles, which can be deployed in a manner substantially similar to the two-needle embodiment described above.

One aspect of the invention relates to a method of delivering fluid compounds to a tubular biological structure. The method comprises inserting a distal portion of an elongated member into the tubular biological structure, deploying one or more hypodermic needles, piercing the interior surface of the tubular biological structure, moving a plunger within a syringe to transfer fluid from the syringe to the tubular biological structure, and withdrawing the hypodermic needles back to their retracted positions.

Another aspect of the invention relates to a method of extracting fluid from a biological structure that is not directly accessible. The method comprises inserting and navigating a distal portion of an elongated member to a desired location of the biological structure, deploying one or more hypodermic needles, piercing the surface of the biological structure, moving a plunger within a syringe to transfer fluid from the biological structure through the hypodermic needles into the syringe, and withdrawing the hypodermic needles back to their retracted positions.

Still another aspect of the invention relates to a method of transferring fluid to or from a biological structure beyond the exterior surface of a tubular biological structure. The method comprises inserting a distal portion of an elongated member into the tubular biological structure, moving the distal portion within the tubular biological structure to a location nearest to the biological structure to be treated, deploying one or more hypodermic needles, piercing the interior surface of the tubular biological structure, piercing the exterior surface of the tubular biological structure, penetrating the biological structure to be treated, moving a plunger within a syringe to transfer fluid between the syringe and the biological structure, and withdrawing the hypodermic needles back to their retracted positions.

### BRIEF DESCRIPTION OF THE DRAWINGS

FIG. 1 illustrates one embodiment of a fluid delivery and extraction device of the present invention.

FIG. 2 is a partial cross-sectional view of the fluid delivery and extraction device of FIG. 1.

FIG. 3 illustrates a distal portion of the device of FIG. 1.

FIG. 4 is a partial cross-sectional view of the distal portion of FIG. 3 taken along line 4-4.

FIG. 5 is a cross-sectional view of the distal portion of FIG. 3 taken along line 5-5 of FIG. 4.

FIG. 6 illustrates the distal portion of FIG. 3 with a pair of hypodermic needles deployed.

FIG. 7 illustrates the distal portion of the device of FIG. 1 inserted into a tubular biological structure.

FIG. 8 illustrates the distal portion of the device of FIG. 1 with a pair of hypodermic needles partially deployed.

FIG. 9 illustrates the distal portion of the device of FIG. 1 with the pair of hypodermic needles piercing the walls of the tubular biological structure.

FIG. 10 illustrates the distal portion of the device of FIG. 1 with the pair of hypodermic needles piercing and extended beyond the exterior surface of a tubular biological structure.

FIG. 11 illustrates the distal portion of FIG. 3 with an anchor hook deployed.

FIG. 12 illustrates the distal portion of FIG. 3 with an angioplasty balloon deployed and inflated.

FIG. 13 illustrates the distal portion of FIG. 3 with a pair of biopsy jaws deployed.

### Detailed Description of the Preferred Embodiment

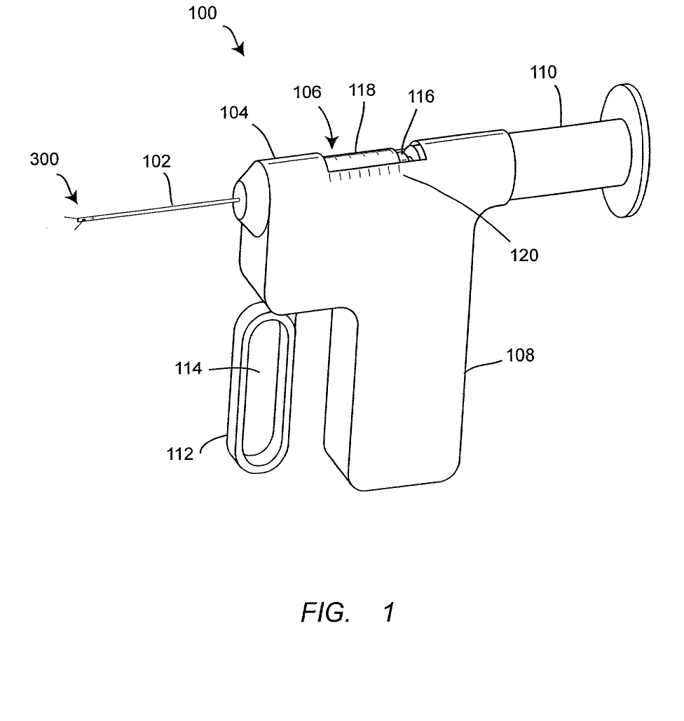

As shown in FIG. 1, an embodiment of a fluid delivery and extraction device 100 comprises a distal portion 300, a shaft 102, a main body 104, a window slot 106, a syringe 116, a luer connector 118, a plunger 110, a handle 108, a trigger actuator 112, a finger aperture 114, and a calibrated index 120. The shaft 102 is preferably flexible to allow it to bend when advanced through internal biological structures, particularly body lumens. The length of the shaft 102 may be modified to accommodate various fluid delivery and extraction applications. The trigger actuator 112 and the luer connector 118 are operatively connected to the distal portion 300 and may be used to remotely manipulate the components of the distal portion 300. The plunger 110 is operatively connected to the syringe 116 and may be used to remotely deliver or extract fluid through the distal portion 300. The window slot 106 enables the volume of fluid transferred to or from the syringe 116 to be visually monitored. In one embodiment, a window slot may advantageously be placed in the luer connector 118 to facilitate the direct viewing of the fluid volume transferred to or from the syringe 116. In another embodiment, an index may advantageously be positioned on the plunger 110 to indicate the fluid volume transferred to or from the syringe 116. When the trigger actuator 112 is moved, the luer connector 118, the syringe 116, and the plunger 110 are free to slide as a unit within the main body 104. The calibrated index markings 120 are positioned on the main body 104 near the window slot 106 to facilitate visual monitoring of the distance to which the luer connector 118 moves when the trigger actuator 112 is moved.

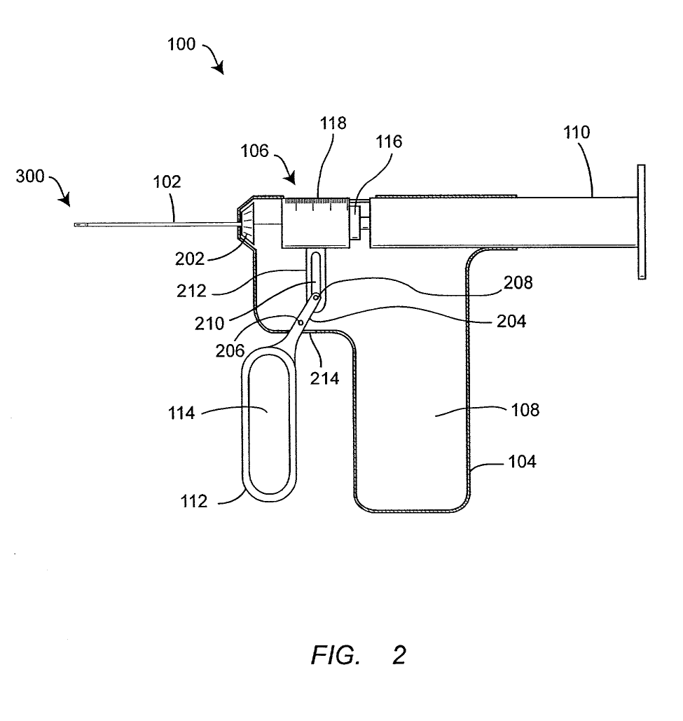

FIG. 2 is a partial cross-sectional view of the fluid delivery and extraction device 100 of FIG. 1. As shown, the proximal ends of a pair of hypodermic needles 308, 308′ are molded into, or otherwise attached to, the luer connector 118. The hypodermic needles 308, 308′ extend distally within the shaft 102 to the distal portion 300. The shaft 102 is attached to the main body 104 by a shaft mount 202. The syringe 116 slides concentrically into and snaps to the luer connector 118. The interface between the syringe 116 and the luer connector 118 is sealed to facilitate the containment and/or transfer of fluids. The plunger 110 slides concentrically within the inner volume of the syringe 116. The interface between the plunger 110 and the syringe 116 is sealed to facilitate the containment of fluid within the syringe 116. The plunger 110 and the syringe 116 may be removed from the luer connector 118 and then slid proximally out of the main body 104, thereby facilitating the transfer of fluid into or out of the syringe 116.

The trigger actuator 112 is operatively connected to the luer connector 118 by an injection actuator 212 and a trigger lever 204. The trigger lever 204 is attached to the main body 104 by a mounting pivot 206. In one embodiment, the mounting pivot 206 may comprise a hole in the trigger lever 204 that accepts a molded cylindrical protrusion from the main body 104. In another embodiment, the mounting pivot 206 may comprise a hole in the trigger lever 204 that accepts a pivot pin that is fixed to the main body 104. The trigger actuator 112 and the trigger lever 204 are free to rotate as a unit about the mounting pivot 206.

The trigger lever 204 is connected to the injection actuator 212 by a sliding pivot 208 which is free to travel along a pivot aperture 210. In one embodiment, the sliding pivot 208 may comprise a molded cylindrical protrusion from the trigger lever 204 which is inserted into the pivot aperture 210. In another embodiment, the sliding pivot 208 may comprise a pivot pin fixed to the trigger lever 204 which is inserted into the pivot aperture 210. When the trigger actuator 112 is moved proximally toward the handle 108, such as when the trigger actuator 112 is squeezed, the trigger lever 204 rotates about the mounting pivot 206. As the trigger lever 204 rotates about the mounting pivot 206, the sliding pivot 208 moves distally, away from the handle 108. While moving distally, the sliding pivot 208 causes the injection actuator 212 and the luer connector 118 to move distally. The injection actuator 212 is rigidly fixed to the luer connector 118. Thus, when the injection actuator 212 moves distally, the luer connector 118, the syringe 116, and the plunger 110 move distally, as well.

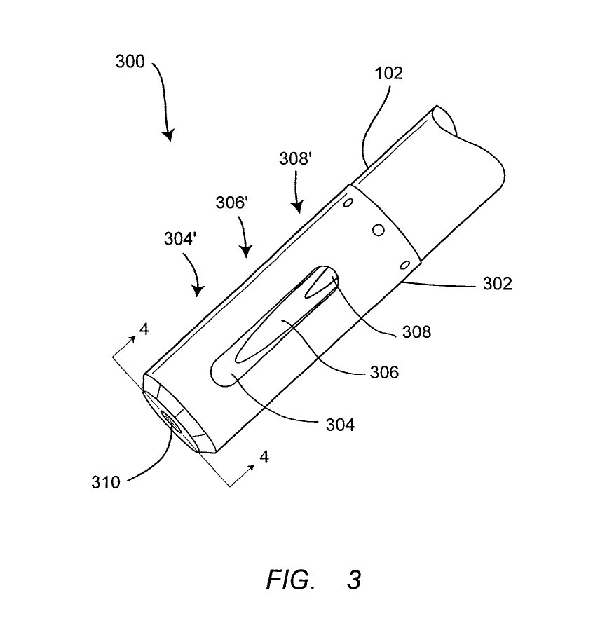

FIG. 3 illustrates the distal portion 300 of the device 100 of FIG. 1. As shown, one embodiment of the distal portion 300 comprises a needle introducer head 302, the pair of hypodermic needles 308, 308′ (not shown), a pair of needle apertures 304, 304′ (not shown), a pair of slanted or curved needle guides 306, 306′ (not shown), and a central lumen 310. When the hypodermic needles 308, 308′ are retracted into the needle apertures 304, 304′ the needles are recessed within the needle introducer head 302 so that the needles do not cause tissue damage upon insertion and retraction of the distal portion 300 from a biological structure.

FIG. 4 is a partial cross-sectional view of the distal portion 300 of FIG. 3 taken along line 4-4. As shown, in one embodiment a steer wire 404 may advantageously be molded into the shaft 102 and the needle introducer head 302 in a lumen 402 extending therethrough. The steer wire 404 advantageously enables remote navigation of the distal portion 300 through internal biological structures, particularly body lumens. More particularly, moving the steer wire proximally or distally enables turning of the distal portion 300 for better navigation.

In another embodiment, a guide wire may be used to guide the placement of the distal portion 300. In this embodiment, a lumen 402 such as described above preferably extends out of the distal end of the needle introducer head. After the guide wire is inserted into the desired body lumen, the shaft 102 is advanced along the guide wire with the guide wire passing within the lumen extending through the distal portion 300. It will be understood that other methods by which the distal portion 300 is remotely navigated through internal biological structures may be employed without detracting from the invention.

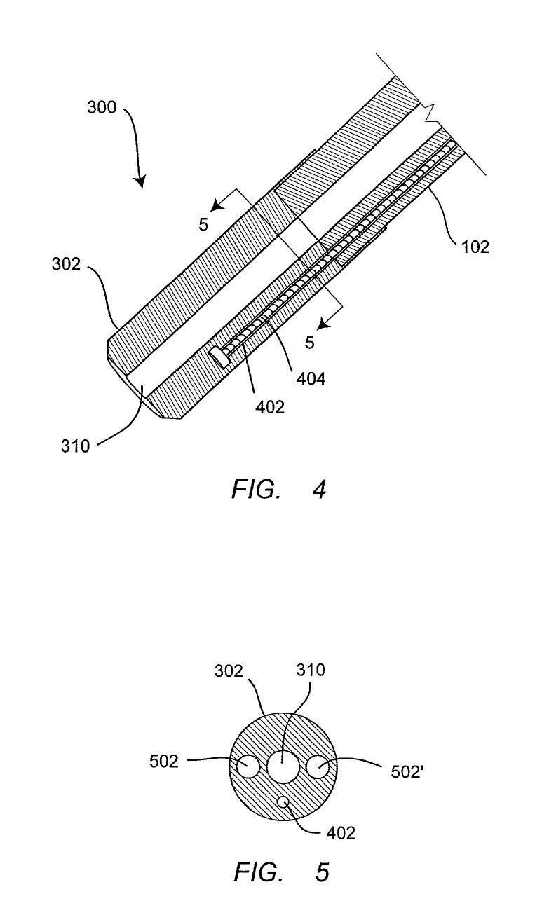

FIG. 5 is a cross-sectional view of the distal portion 300 of FIG. 4 taken along line 5-5 without showing the needles or other components therein. As shown, in one embodiment the needle introducer head 302 is formed with the central lumen 310, a steer wire lumen 402, and a pair of needle lumens 502, 502′. The steer wire lumen 402 facilitates the installation and use of the steer wire 404. The needle lumens 502, 502′ house and guide the hypodermic needles 308, 308′ through the needle introducer head 302. It is to be understood that the shaft 102 possesses the same number of lumens, having the same respective diameters, as does the needle introducer head 302. It is to be further understood that the needle introducer head 302 is fastened to the end of the shaft 102 in such a manner that the lumens in the needle introducer head 302 are aligned with the respective lumens in the shaft 102. Those of ordinary skill in the art will recognize that greater or fewer lumens of varying diameters may advantageously be formed within the needle introducer head 302 and the shaft 102 without detracting from the invention.

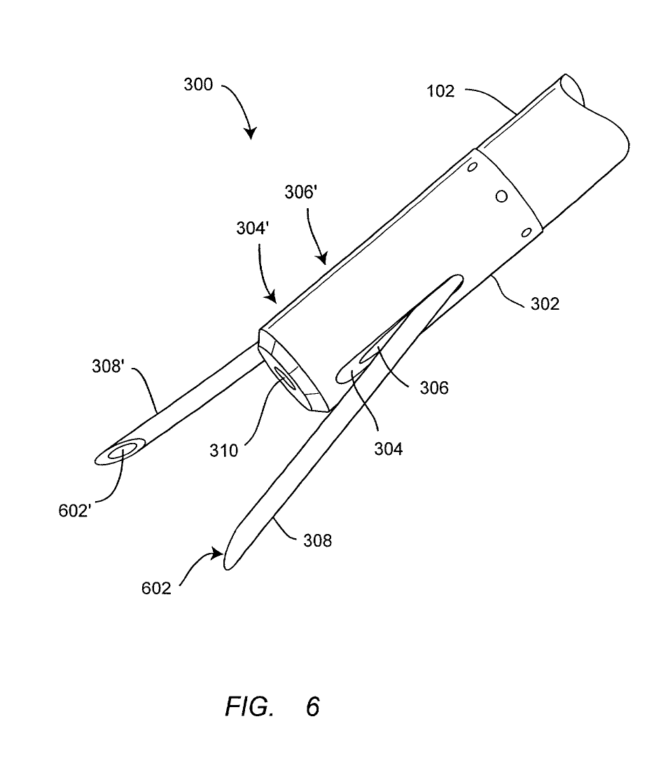

FIG. 6 illustrates the distal portion 300 of the device 100 of FIG. 1 with the hypodermic needles 308, 308′ deployed outwardly from their recessed position within the needle introducer head 302. The hypodermic needles 308, 308′ may be advanced from a recessed position within the needle introducer head 302 to a distally extended position by squeezing the trigger actuator 112. When the two hypodermic needles 308, 308′ are moved distally, the curved needle guides 306, 306′ guide the hypodermic needles 308, 308′ out of the needle apertures 304, 304′ at an angle relative to the axis of the needle introducer head 302. The hypodermic needles 308, 308′ have central injection lumens 602, 602′ to facilitate the delivery and extraction of fluids. It will be appreciated that other hypodermic needles of differing lengths, exterior diameters, and internal diameters may be employed without detracting from the invention. Furthermore, although the distal portion 300 of the device 100 is illustrated with both hypodermic needles 308, 308′ deployed simultaneously (FIG. 6), it will be understood that the device 100 may also be configured to deploy the hypodermic needles 308, 308′ individually without detracting from the invention.

The fluid delivery and extraction device 100 of FIG. 1 may be used to medically treat a variety of biological structures. In general, a physician inserts the distal portion 300 into a cavernous or tubular structure within a patient to give injections to at least one tissue portion. In one embodiment, the steer wire 404 may be used to navigate the distal portion 300 within the patient. In another embodiment, a guide wire may be used to direct the distal portion 300 as it moves within the patient.

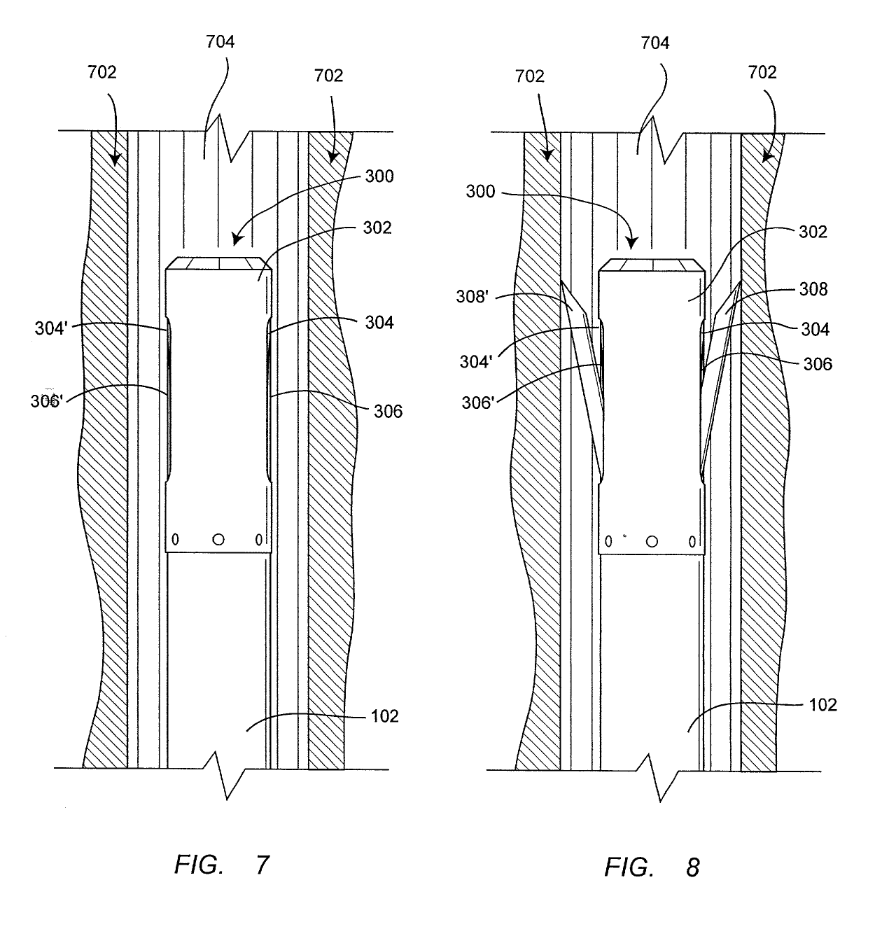

Referring to FIG. 7, once the distal portion 300 is positioned at the desired location within a tubular biological structure 704, such as a body lumen, the physician squeezes the trigger actuator 112 to deploy the hypodermic needles 308, 308′. FIG. 8 illustrates the distal portion 300 with the pair of hypodermic needles 308, 308′ beginning to advance from their recessed positions within the needle introducer head 302.

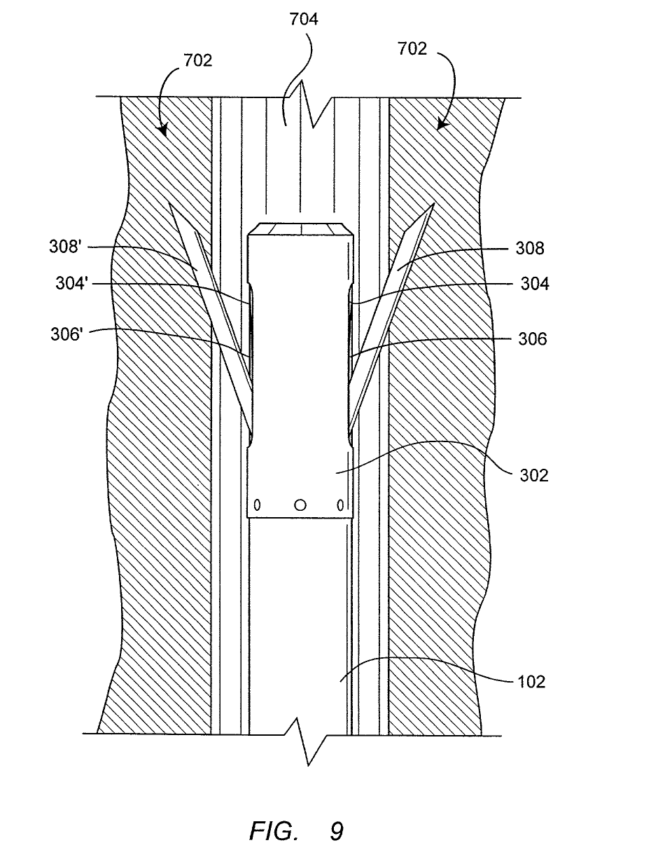

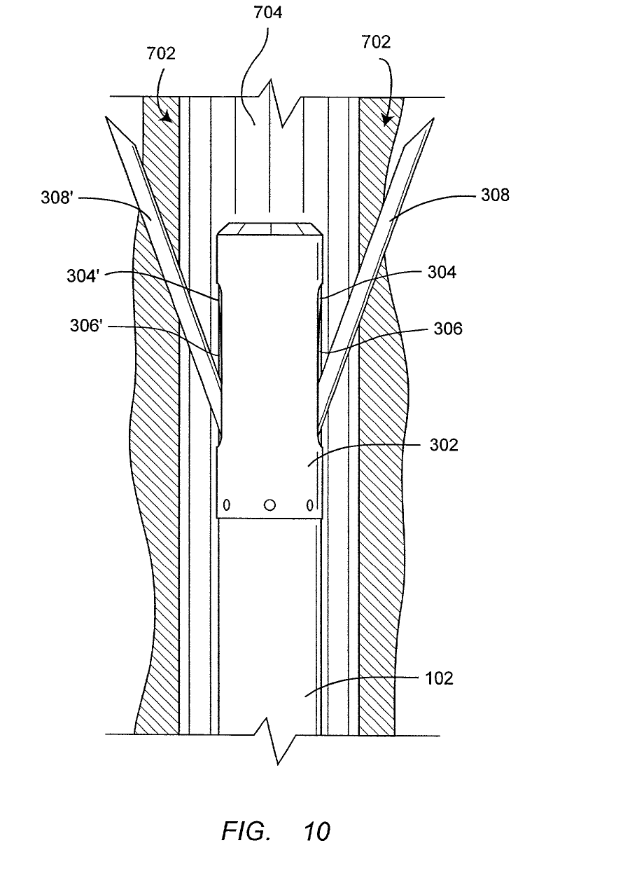

As shown in FIG. 9, when the hypodermic needles 308, 308′ advance from their recessed positions within the needle introducer head 302, the hypodermic needles 308, 308′ pierce the interior surface of walls 702 of the tubular biological structure 704. If desired, force may be applied to the shaft 102 in order to move the needle introducer head 302 distally so as to assist the hypodermic needles 308, 308′ in piercing the walls 702. In one embodiment, the length of the hypodermic needles 308, 308′ and/or the diameter of the needle introducer head 302 are selected such that the hypodermic needles 308, 308′ penetrate well beyond the interior surface of walls 702. The physician may view the calibrated index markings 120 to monitor the distance to which the hypodermic needles 308, 308′ extend beyond the needle introducer head 302.

Once the hypodermic needles 308, 308′ have penetrated to the desired distance within the walls 702, the plunger 110 may be moved distally to deliver a volume of fluid from the syringe 116 to the tubular biological structure 704 via the hypodermic needles 308, 308′. In addition, if fluid is to be extracted from the tubular biological structure 704, the plunger 110 may be moved proximally to draw a volume of fluid from the walls 702 into the syringe 116 via the hypodermic needles 308, 308′. The volume of fluid transferred to or from the syringe 116 may be viewed through the window slot 106 on the main body 104. Once the desired volume of fluid has been transferred, the physician moves the trigger actuator 112 distally to move the hypodermic needles 308, 308′ to their retracted positions within the needle introducer head 302 as shown in FIG. 7. The physician then withdraws the distal portion 300 from the patient. One preferred method involves the use of device 100 to inject fluid compounds into the walls of a blood vessel. Other methods may, for example, involve injecting fluid compounds into the walls of a fallopian tube or other body lumen.

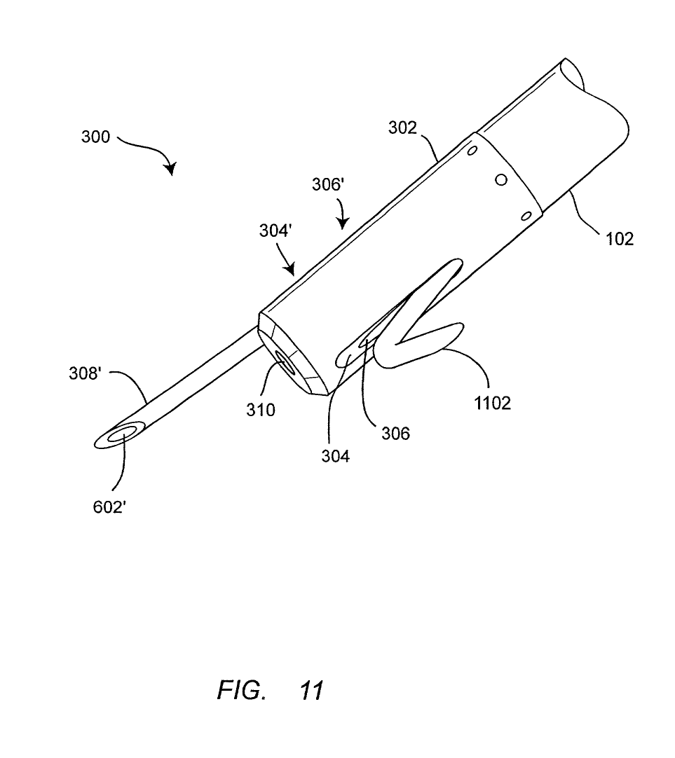

Although the above-discussed procedure pierced the walls 702 without penetrating the exterior surface of the tubular biological structure 704, it will be understood that the fluid delivery and extraction device 100 may also be configured to pass the hypodermic needles 308, 308′ completely through the walls 702 so as to penetrate the exterior surface of the tubular biological structure 704. FIG. 10 illustrates the distal portion 300 of the device 100 of FIG. 1 with both the hypodermic needles 308, 308′ extending through the exterior surface of the walls 702 of the tubular biological structure 704. When the physician squeezes the trigger actuator 112, the hypodermic needles 308, 308′ advance distally from their recessed positions within the needle introducer head 302 and pierce the walls 702 of the tubular biological structure 704. As the hypodermic needles 308, 308′ advance, the physician may push the distal portion 300 distally to cause the hypodermic needles 308, 308′ to be driven into the tissue and through the walls 702. The physician may view the calibrated index 120 to monitor the distance to which the hypodermic needles 308, 308′ extend beyond the needle introducer head 302. The physician then pushes the plunger 110 distally to transfer a volume of fluid from the syringe 116 to the patient, or pulls the plunger 110 proximally to draw a volume of fluid from the patient into the syringe 116. The volume of fluid transferred to or from the syringe 116 may be viewed through the window slot 106. When the desired volume of fluid has been transferred, the physician withdraws the hypodermic needles 308, 308′ into the needle introducer head 302 and removes the distal portion from the patient. One preferred use of the device 100 is to inject fluid compounds into the prostate gland from within the urethra.

FIG. 11 illustrates one embodiment of the distal portion 300 of the device 100 in which an anchor hook 1102 is deployed. The anchor hook 1102 serves as a means by which the distal portion 300 can be anchored or approximated to desired locations within biological structures, particularly body lumens. In one embodiment, the anchor hook 1102 replaces the needle 308, and consequently is located on the opposite side of the needle introducer head 302 from the needle 308′. In another embodiment, the anchor hook 1102 is installed in addition to the hypodermic needles 308, 308′. With this embodiment, the needle introducer head 302 and the shaft 102 are formed with an additional lumen dedicated to housing and guiding the anchor hook 1102; the needle introducer head 302 is fabricated with an additional needle aperture 304 and curved needle guide 306. The anchor hook 1102 may advantageously be operatively connected to controls located on the main body 104 thereby facilitating the remote operation of the anchor hook 1102 within biological structures.

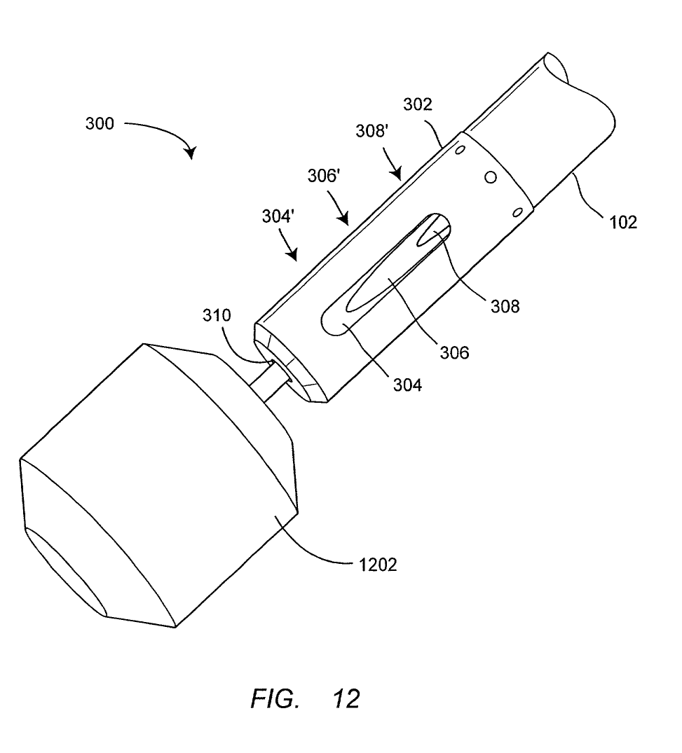

FIG. 12 illustrates another embodiment of the distal portion 300 of the device 100, in which a balloon 1202 is deployed and inflated through the central lumen 310 and then inflated. In one embodiment, the balloon 1202 may be used to anchor or approximate the distal portion 300 to desired locations within biological structures, particularly body lumens. In another embodiment, the balloon 1202 may be an angioplasty balloon inflated in order to dilate ischemic tissue or a stenosis as is commonly performed in angioplasty procedures. The balloon 1202 may advantageously be operatively connected to controls located on the main body 104 thereby facilitating the remote operation of the angioplasty balloon within biological structures.

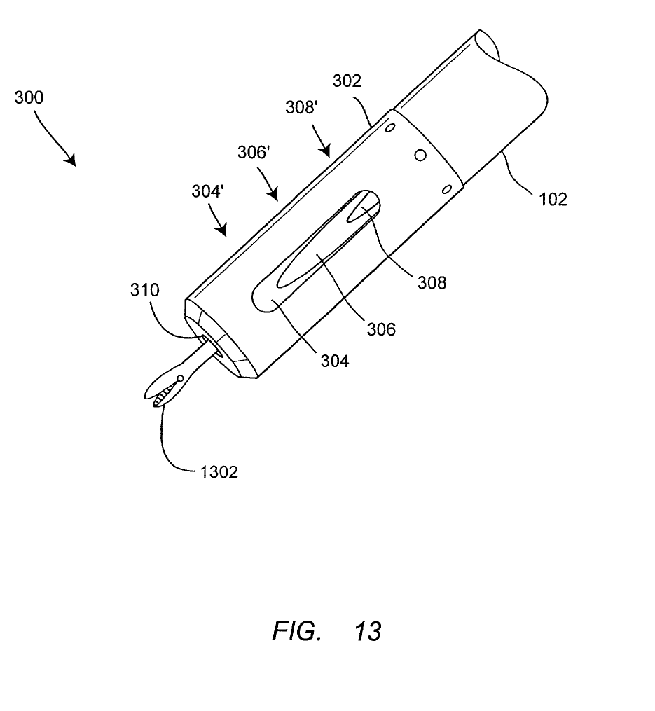

FIG. 13 illustrates yet another embodiment of the distal portion 300 of the device 100, in which a pair of biopsy jaws 1302 is deployed through the central lumen 310. In one embodiment, the biopsy jaws 1302 may advantageously be used to perform biopsy procedures on biological structures that are not directly accessible. In another embodiment, the biopsy jaws 1302 may advantageously be used to anchor or approximate the distal portion 300 to desired locations within biological structures, particularly body lumens. The biopsy jaws 1302 may advantageously be operatively connected to controls located on the main body 104 thereby facilitating the remote operation of the biopsy jaws 1302 within biological structures.

It will be appreciated that the central lumen 310 may be used to provide devices other than the balloon and biopsy jaws described above distal to the distal portion 300.

In still another embodiment of the device 100, the distal portion 300 has four hypodermic needles 308 that deploy through four needle apertures 304. The operation of this four-needle, four-aperture embodiment is substantially similar to the operation of the two-needle, two-aperture embodiment described above with reference to FIGS. 1-13. In yet another embodiment of the device 100, the distal portion 300 has six hypodermic needles 308 that deploy through six needle apertures 304. In yet another embodiment of the device 100, the distal portion 300 has eight hypodermic needles 308 that deploy through eight needle apertures 304. Devices with other numbers of needles are also contemplated.

While embodiments and applications of this invention have been shown and described, it will be apparent to those skilled in the art that various modifications are possible without departing from the scope of the invention. It is, therefore, to be understood that within the scope of the appended claims, this invention may be practiced otherwise than as specifically described.

What is claimed is:

1. A fluid delivery and extraction device, comprising:
an elongate shaft having a proximal end and a distal end;
at least one lumen extending through the elongate shaft;
at least one needle having a proximal end and a distal end extending at least partially through a lumen in the shaft, the needle being moveable from a first position within the elongate shaft to a second position extending away from the elongate shaft;
a lumen extending through the at least one needle for delivering fluids therethrough in either a proximal to distal direction or in a distal to proximal direction; and
a syringe in fluid communication with the proximal end of the needle, the syringe being adapted to either deliver fluid through the needle or withdraw fluid from the needle.

2. The device of claim 1, comprising two needles extending at least partially through the shaft and being moveable from a first position within the elongate shaft to a second position extending away from the elongate shaft, each needle having a fluid delivery lumen extending therethrough.

3. The device of claim 2, wherein the two needles extend through a first and second needle lumen, respectively, within the elongate shaft.

4. The device of claim 1, further comprising a balloon movable past the distal end of the elongate shaft.

5. The device of claim 1, further comprising a pair of jaws moveable past the distal end of the elongate shaft.

6. The device of claim 1, further comprising a central lumen separate from the lumen having the at least one needle.

7. The device of claim 1, further comprising a steer wire within the elongate shaft.

8. The device of claim 1, further comprising a guidewire lumen.

9. The device of claim 1, further comprising a housing receiving the proximal end of the shaft.

10. The device of claim 9, wherein the syringe is inside the housing.

11. The device of claim 9, further comprising a trigger actuator for moving the at least one needle from the first position to the second position.

12. The device of claim 9, further comprising calibrated index markings on the housing for determining the distance the at least one needle moves from the first position to the second position.

13. A fluid delivery and extraction device, comprising:
a main body having a handle and a trigger actuator;
a syringe located within the body and operatively connected to the trigger actuator;
a plunger operatively connected to the syringe, the plunger being moveable in a distal direction into the syringe for delivering fluid and in a proximal direction out of the syringe for extracting fluid;
a pair of hollow needles each having a proximal end and a distal end and a lumen in fluid communication with the syringe, the needles extending in a distal direction away from the syringe and the main body; and
an elongate shaft extending distally away from the main body and having at least one lumen for housing the hollow needles;
wherein the trigger actuator when moved toward the handle causes the needles, the syringe and the plunger to move in a distal direction with respect to the body, the distal movement of the needles causing the distal ends of the needles to move out of the shaft.

14. The device of claim 13, wherein the shaft includes a pair of needle lumens, each lumen housing one of the two needles.

15. The device of claim 14, wherein the needle lumens each has a needle guide for directing the needles at an angle outside of the shaft.

16. The device of claim 13, further comprising a central lumen for delivering devices therethrough distal to the distal end of the shaft.

17. A device for communicating fluids, comprising:
a shaft having a proximal end and a distal end and at least one lumen extending therethrough;
at least one needle having a proximal end and a distal end extending through the shaft, the needle being hollow for communicating fluids therethrough between a proximal opening and a distal opening, the needle being moveable from a first position wherein the distal end is within the shaft to a second position wherein the distal end is outside of the shaft.

18. The device of claim 17, wherein the distal end when in the second position is at an angle to the longitudinal axis of the shaft.

19. The device of claim 17, further comprising a source of negative pressure in fluid communication with the proximal opening.

20. The device of claim 17, further comprising a source of positive pressure in fluid communication with the proximal opening.

21. The device of claim 17, wherein the shaft includes at least two lumens extending therethrough.

22. The device of claim 21, comprising a pair of hollow needles, each of said needles being provided in one of the two lumens.

23. The device of claim 21, further comprising an anchor hook extending through one of the two lumens.

24. The device of claim 17, wherein the lumen terminates at a distal end in a sidewall of the shaft.

25. The device of claim 21, wherein at least one of the at least two lumens extends entirely to the distal end of the shaft.

26. The device of claim 21, further comprising a balloon catheter extending through one of the at least two lumens.

27. The device of claim 21, further comprising a pair of jaws extending through one of the at least two lumens.

28. The device of claim 21, further comprising a steer wire extending through one of the at least two lumens.

29. The device of claim 21, wherein one of the at least two lumens is a guidewire lumen.

30. The device of claim 21, comprising at least three lumens extending through the shaft.

31. The device of claim 21, wherein two hollow needles extend through the shaft in two of the at least three lumens.

32. The device of claim 31, wherein the two lumens containing the hollow needles are provided on opposite sides of the shaft, with the third lumen being a central lumen therebetween.

33. The device of claim 32, wherein the two lumens containing the needles further include a needle guide for directing the needles at an angle to the longitudinal axis of the shaft when the distal ends of the needles are moved from their first position to their second position.

34. The device of claim 30, comprising at least four lumens extending through the shaft.

35. A method for delivering fluid to a tubular biological structure, comprising:
delivering a distal portion of an elongated member into the tubular biological structure;
deploying one or more needles from within the elongated member to outside the elongated member;
piercing an interior surface of the biological structure with the one or more needles; and
extracting fluid from the biological structure through a lumen in the one or more needles.

36. The method of claim 35, wherein piercing the interior surface of the biological structure with the one or more needles further comprises piercing through to the exterior surface of the biological structure.

37. The method of claim 35, wherein extracting fluid from the biological structure comprises moving a plunger within a syringe connected to the one or more needles.

38. The method of claim 35, further comprising, while piercing the interior surface of the biological structure, moving the elongated member distally to assist in piercing the interior surface.

39. The method of claim 35, comprising deploying two needles simultaneously.

40. The method of claim 35, comprising deploying two needles sequentially.

41. The method of claim 35, wherein the elongated member is delivered over a guidewire.

42. The method of claim 35, further comprising anchoring the elongated member prior to piercing the interior surface.

43. The method of claim 42, wherein anchoring is achieved with an anchoring hook deployed through the elongated member.

44. The method of claim 42, wherein anchoring is achieved with an inflatable balloon deployed through the elongated member.

45. The method of claim 42, wherein anchoring is achieved with a pair of jaws deployed through the elongated member.

46. The method of claim 35, further comprising delivering an angioplasty balloon through the elongated member.

47. A method for extracting fluid from a tubular biological structure, comprising:
delivering a distal end portion of an elongated member into the tubular biological structure;
deploying one or more needles from within the elongated member to outside the elongated member;
piercing an interior surface of the biological structure with the one or more needles; and
delivering fluid to the biological structure through a lumen in the one or more needles.

48. The method of claim 47, wherein piercing the interior surface of the biological structure with the one or more needles further comprises piercing through to an exterior surface of the biological structure.

49. The method of claim 47, wherein delivering fluid to the biological structure comprises moving a plunger within a syringe connected to the one or more needles.

50. The method of claim 47, further comprising, while piercing the interior surface of the biological structure, moving the elongated member distally to assist in piercing the interior surface.

51. The method of claim 47, comprising deploying two needles simultaneously.

52. The method of claim 47, comprising deploying two needles sequentially.

53. The method of claim 47, wherein the elongated member is delivered over a guidewire.

54. The method of claim 47, further comprising anchoring the elongated member prior to piercing the interior surface.

55. The method of claim 54, wherein anchoring is achieved with an anchoring hook deployed through the elongated member.

56. The method of claim 54, wherein anchoring is achieved with an inflatable balloon deployed through the elongated member.

57. The method of claim 54, wherein anchoring is achieved with a pair of jaws deployed through the elongated member.

58. The method of claim 47, further comprising delivering an angioplasty balloon through the elongated member.
